(19) United States
(12) Patent Application Publication
(10) Pub. No.: US 2022/0355033 A1
(43) Pub. Date: Nov. 10, 2022

(54) MEDICAL INJECTION SYSTEM AND METHOD

(71) Applicant: Preci Health SA, Neuchåtel (CH)

(72) Inventors: Philippe Margairaz, La Chaux-de-Fonds (CH); Carlos Da Silva, Fleurier (CH); Luc Maffli, Fribourg (CH); Alain Jaccard, Ste-Croix (CH)

(73) Assignee: Preci Health SA, Neuchåtel (CH)

(21) Appl. No.: 17/621,288

(22) PCT Filed: Jun. 22, 2020
(86) PCT No.: PCT/IB2020/055874

§ 371 Date: Dec. 21, 2021

Related U.S. Application Data
(60) Provisional application No. 62/864,572, filed on Jun. 21, 2019.

(51) Int. Cl. A61M 5/20; A61M 5/32

(52) U.S. Cl. CPC A61M 5/2033; A61M 5/3234; A61M 2005/208

(57) ABSTRACT

A fluid dispensing device adapted for intramuscular injection into a living organism is provided. The device has an energy storage arrangement providing energy in at least two bursts. The first burst is provided from a first energy source and is that which is necessary to pierce the skin of the living organism and insert the needle to the injection site. The second burst is provided by a second energy source and is that which is necessary to pump the fluid out of the reservoir and into the living organism. Optionally, the remaining energy is used to retract the needle out of the living organism. Together with a prescribed operating sequence of the device, use of the two sources of energy contained in the device is optimized to provide the at least two bursts of energy.

### CROSS REFERENCE TO RELATED APPLICATIONS

This application claims the benefit of U.S. Provisional Application No. 62/864,572, filed Jun. 21, 2019, entitled: MEDICAL INJECTION SYSTEM AND METHOD, which is incorporated herein by reference in its entirety.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND OF THE INVENTION

This invention relates to devices allowing to bring the fluids, optionally including medicine, into a living organism in a controlled and automatized manner.

### SUMMARY OF THE INVENTION

A fluid dispensing device is adapted to dispense a measured amount of fluid, preferably into the muscle, typically intramuscular, of a living organism is provided. The fluid dispensing device is also adapted to dispense a measured amount of fluid intracutaneous or subcutaneous to a living organism. The device has an energy storage arrangement providing energy in at least two bursts. The first burst is provided from a first energy source and is that which is necessary to pierce the skin of the living organism and insert the needle to the injection site. The second burst is provided by a second energy source and is that which is necessary to pump the fluid out of the reservoir and into the living organism and to retract the needle out of the living organism. Together with a prescribed operating sequence of the device, use of the two sources of energy contained in the device is optimized to provide the at least two bursts of energy.

The fluid dispensing device has a main housing enclosing the operative components of the device, a fluid reservoir located within the main housing, an injection assembly including a needle, and a trigger mechanism. The fluid reservoir mentioned in the instant description is typically a cartridge, although other reservoirs can be used. The needle has a first end and a second end and is adapted for interfacing, on the first end, with a septum disposed in the reservoir containing the fluid to be dispensed, and at a second end thereof, for inserting into a living organism. The mechanism contains the energy necessary to insert the needle into the living organism, dispense the fluid, and retract the needle, and ensures the completion of an operational cycle without any other action from the user other than triggering (which is typically accomplished while holding the device firmly in place). The first and second ends of the needle pierce respective surfaces while translating together in a direction parallel to the skin for the first end of the needle and substantially non orthogonal to the surface of the living organism's skin for the second end of the needle. Once the septum is pierced, the needle is in fluid communication with the fluid reservoir and the device expels the fluid through the needle into the living organism.

The invention is disclosed herein in three principal embodiments. The second and third embodiments are alternative embodiments of the first embodiment. Particularities of the second embodiment are shown in FIGS. 8A, 8B, 10A, 10B and 12. Otherwise, if, for certain operational steps, no particular figure of the second embodiment specifically discloses this feature or step, the corresponding figure of the first embodiment is representative of all embodiments to support this feature or step in the other embodiments. The first and the second embodiment differ mainly in their mechanical proportions. The first embodiment is intended for intramuscular application, i.e. performing an injection typically of 2-3 cm into the patient's soft tissue, while the second embodiment is intended for subcutaneous application, i.e. performing an injection typically 1-2 mm into the patient's soft tissue. Hence, the first embodiment typically includes a long needle, the long needle having a length adapted to penetrate 2-3 cm into the patient's tissue, while the second embodiment typically includes a short needle, the short needle having a length adapted to penetrate 1-2 mm into the patient's soft tissue. Each of the first and the second embodiment includes a cartridge. The cartridge of the first embodiment (containing about 1 ml of fluid) is adapted to contain concentrated drugs, while the cartridge of the second embodiment (containing about 3 ml of fluid) is adapted to contain drugs with standard concentration.

In a third embodiment, the fluid dispensing device is retained (via the user, an elastic band or an adhesive or adhesive pad) against the skin of the living organism and may be manually, automatically or remotely actuated to inject a fluid into the living organism. Such features of the third embodiment may readily be integrated in the first and second embodiments.

In a fourth embodiment, devices are stacked (see FIG. 2). Such feature of the fourth embodiment may also readily be integrated in the first three embodiments.

In a fifth embodiment, instead of a replaceable cartridge, the reservoir can be filled with a fluid into a dedicated reservoir in the device itself, such feature also easily being integrated into the other embodiments described herein.

It is an object of the invention to provide an easy to use, user friendly device that allows any person, even the patients themselves, to perform an injection of a fluid, of particular interest, a COVID-19 vaccine, without the intervention of medical personnel, thereby reducing the burden on world healthcare systems.

It is another object of the invention to provide a user friendly device that allows a patient to inject himself without requiring the user to make an aggressive gesture like stabbing himself.

It is another object of the invention to provide a device that can step up to the challenge of new molecular therapeutic innovations such as high viscosity fluids whose characteristics otherwise limit administration options.

In an advantage, the embodiment of the invention using more than one needle enables simultaneous injection of several smaller doses of fluid into a living organism instead of one larger dose, generating less local tissue damage and therefore reducing the pain of the injection for the patient.

It is another object of the invention to provide a device with and extended product life cycle.

It is another object of the invention to provide a device that retracts the needle to prevent inadvertent harm that might be caused by an exposed needle.

It is another object of the invention to provide the option of injecting at an angle as the needle passes through a mandrel.

It is another object of the invention to prevent unsanitary or perhaps inadvertent reuse of the device, thus providing a safer means of injecting a fluid.

### BRIEF DESCRIPTION OF THE DRAWINGS

FIG. 1 is a perspective view of a first embodiment of a device according to the invention.

FIG. 2A is a perspective view of a second embodiment of a device according to the invention.

FIG. 2B is a view of two devices according to the second embodiment of the invention attached together.

FIG. 3 is a partially exploded perspective view of the first embodiment of the invention while being prepared for use.

FIG. 4 is a partially exploded perspective view of the second embodiment of the invention while being prepared for use.

FIG. 5A is a partial cross-sectional view of a mechanism according to the invention.

FIG. 5B is a cross-sectional view of the mechanism of the invention.

FIG. 6A is a partial perspective view of the needle subassembly according to the invention.

FIG. 6B is a partial perspective view of the insertion subassembly and the needle subassembly according to the invention.

FIG. 6C is a partial perspective view of the subassemblies of FIG. 6B, from a first rearward perspective.

FIG. 6D is a partial perspective view of the subassemblies of FIG. 6B, from below.

FIG. 7A is a perspective view of the injection subassembly according to the invention.

FIG. 7B is a cross-sectional view of the injection subassembly of FIG. 7A showing the internal reservoir subassembly according to the invention.

FIG. 8A is a cross-sectional view of an alternative embodiment of the injection subassembly as shown in FIGS. 6A and 6B according to the invention.

FIG. 8B is another cross-sectional view of the alternative embodiment of FIG. 8A.

FIG. 9A is a cross-section view of the mechanism of the invention at the step 6 of its use.

FIG. 9B to 9D are cross-sectional views of the mechanism of the invention at the step 7 of its use.

FIGS. 10A-10B are cross-sectional views of the mechanism of the invention of an alternative embodiment at step 7 of its use.

FIG. 11 is a cross-sectional view of the mechanism of the invention at the step 8 of its use.

FIG. 12 is a cross-sectional view of the mechanism of the invention of an alternative embodiment at the step 8 of its use.

FIG. 13 is a cross-sectional view of the mechanism of the invention at the step 9 of its use.

FIGS. 14A-14B are cross-sectional views of the mechanism of the invention at the step 10 of its use.

FIG. 15 is a side view of the needle.

FIG. 16 is a cross-sectional view of an alternative embodiment.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’. ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as they are exemplary in nature, serving to describe the best mode of the invention known the inventors as of the filing date hereof. Consequently, changes may be made in the arrangement and/or function of any of the elements described in the exemplary embodiments disclosed herein without departing from the spirit and scope of the invention.

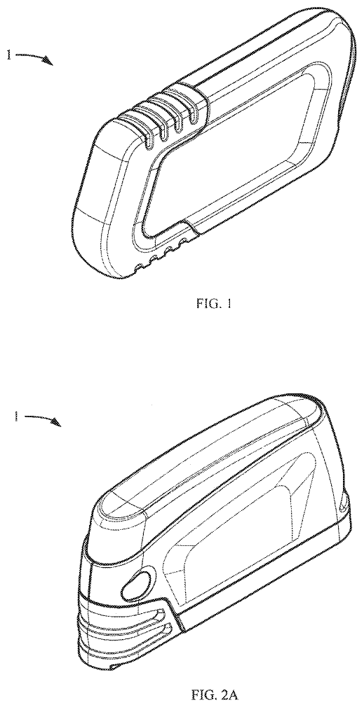

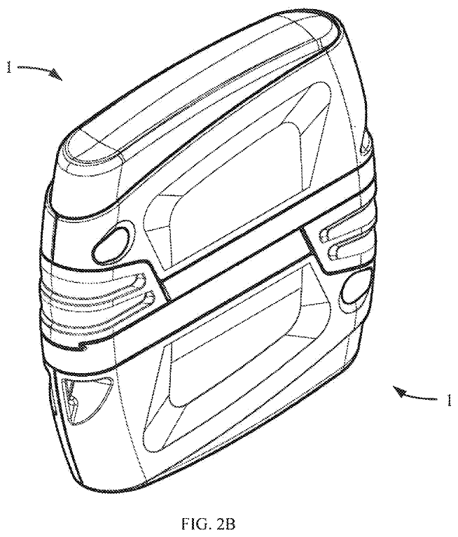

Referring now to FIGS. 1, 2A and 2B, the device 1 of the invention has a narrow profile shape that is easy to carry in a purse or a pocket. Optionally, a pair of devices may be provided attached together for convenience.

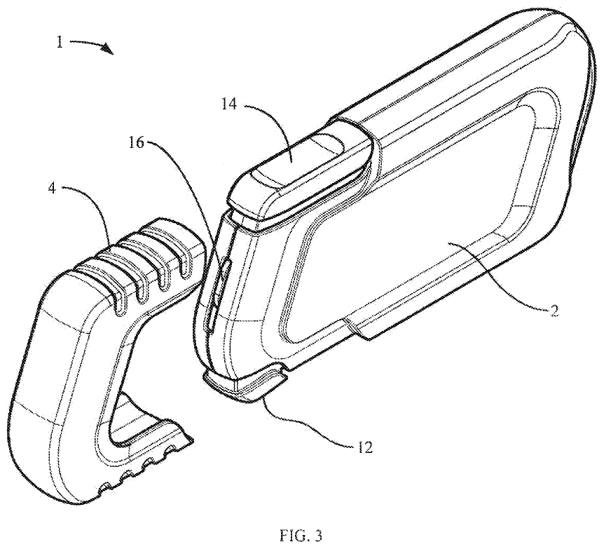

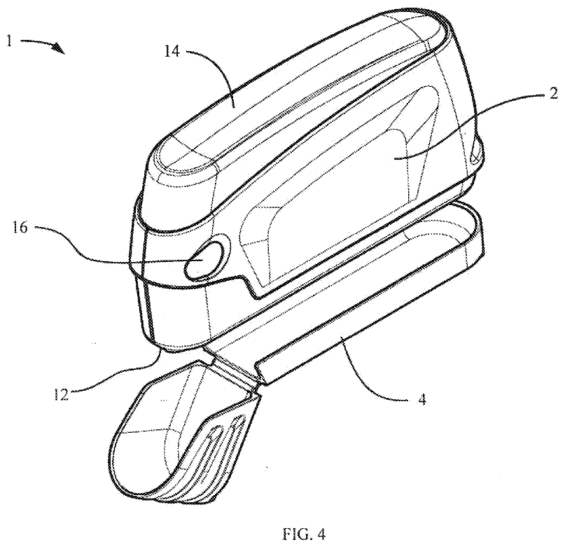

Referring now to FIGS. 3 and 4, the device 1 has a main body 2 and a cover 4 that the user removes in order to have access to the push button 14. An indicator 16 allows the user to check whether the device has already been used or is available for use. Once the cover 4 has been removed, the user applies the device main body 2 against the skin of a living organism. An optional pressure-sensitive safety release mechanism 12 releases the push button 14 only when the pressure applied on the skin of the living organism is above a predefined threshold to ensure correct disposition/retention for optimal operation of the device prior to allowing the push button 14 to be pressed for triggering.

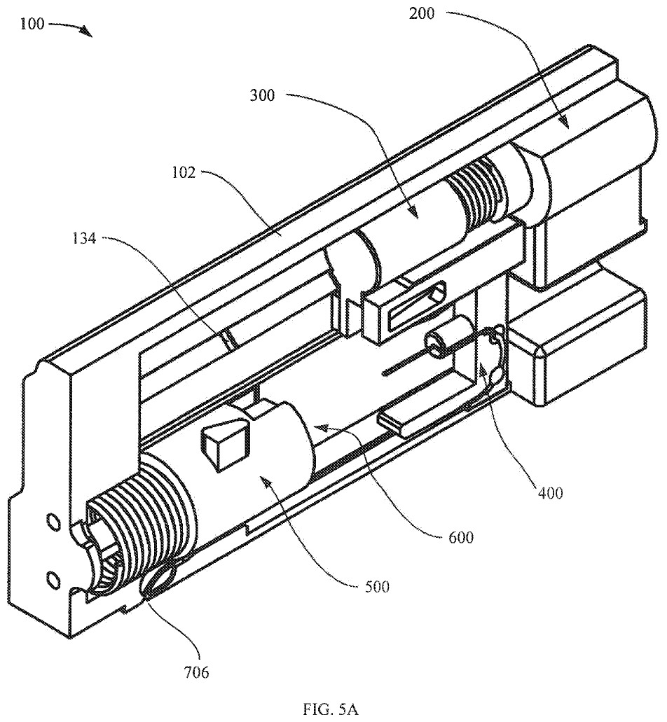

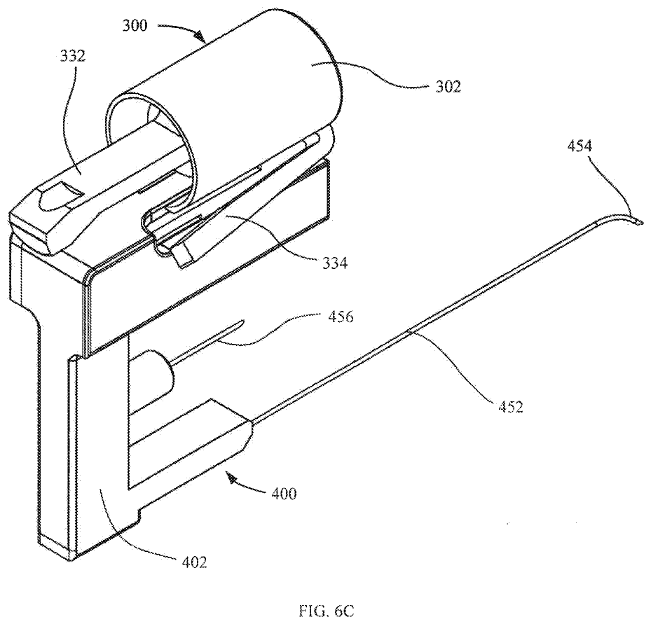

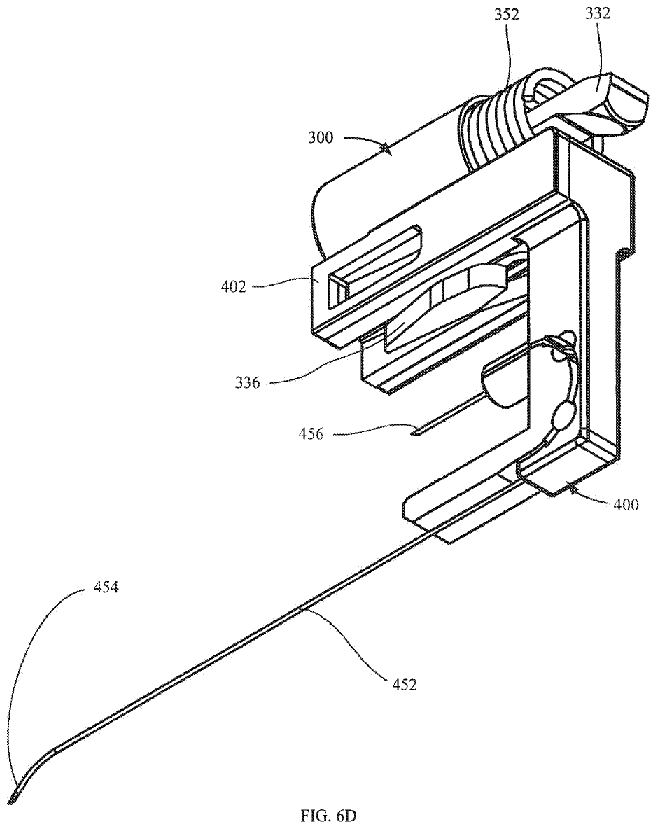

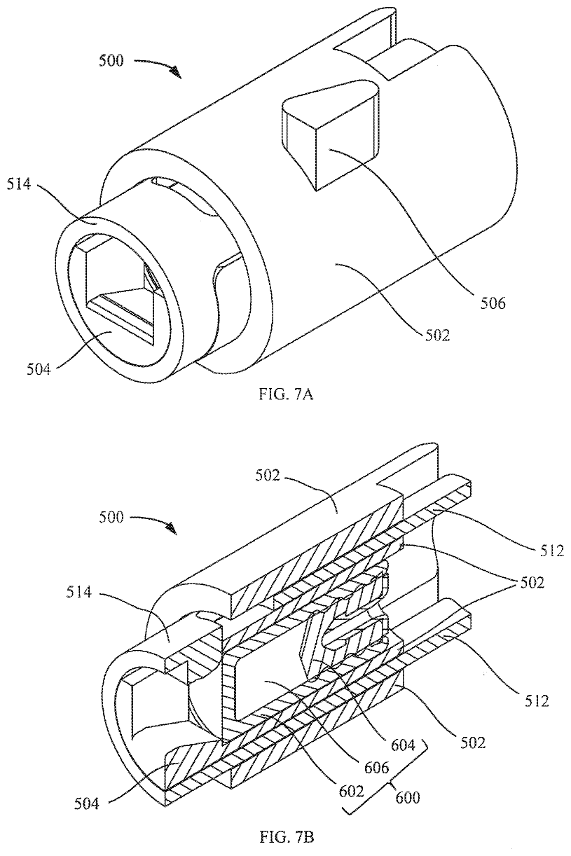

Referring now to FIGS. 5A and 5B, the mechanism 100 of the medical device 1 of the invention is made up of five main subassemblies which are contained in the main structure 102; the trigger mechanism 200, the insertion subassembly 300, the needle subassembly 400, the dispensing subassembly 500 and the reservoir subassembly 600 (shown in FIG. 7B). The main structure may be made of injected polymer, or any other suitable material.

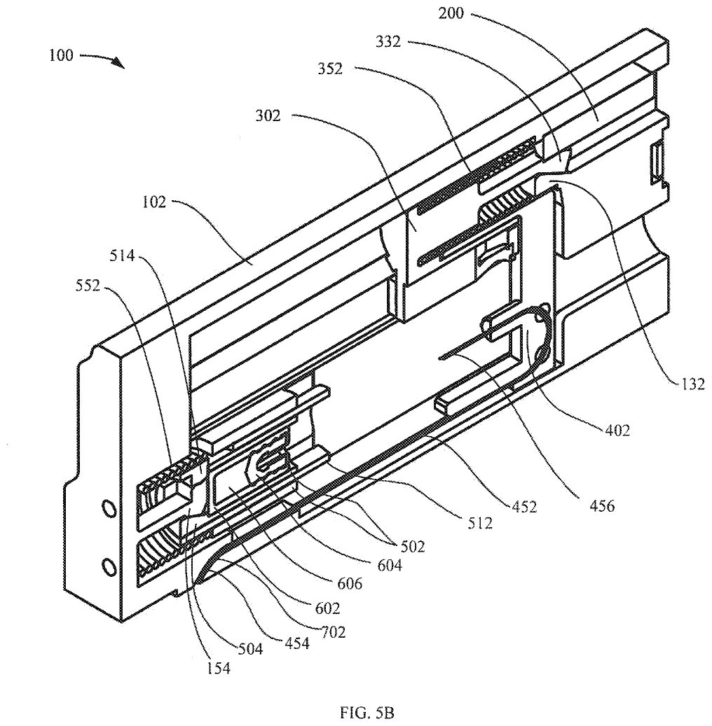

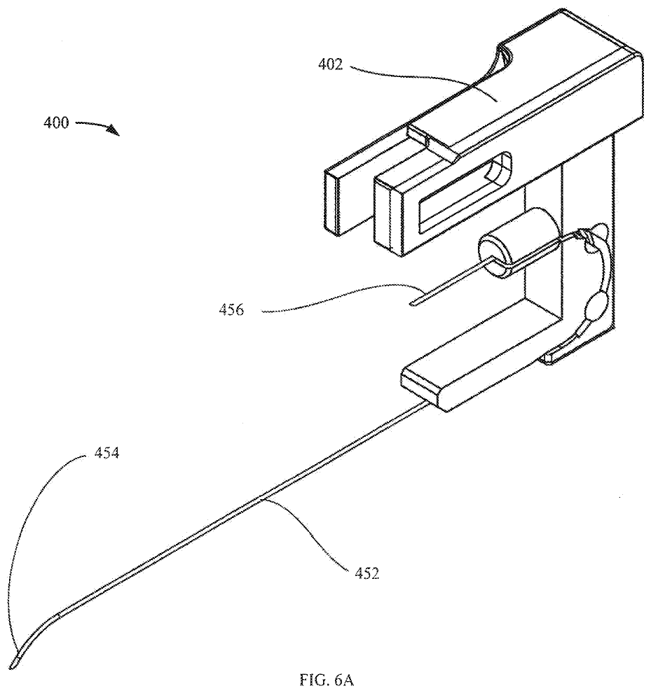

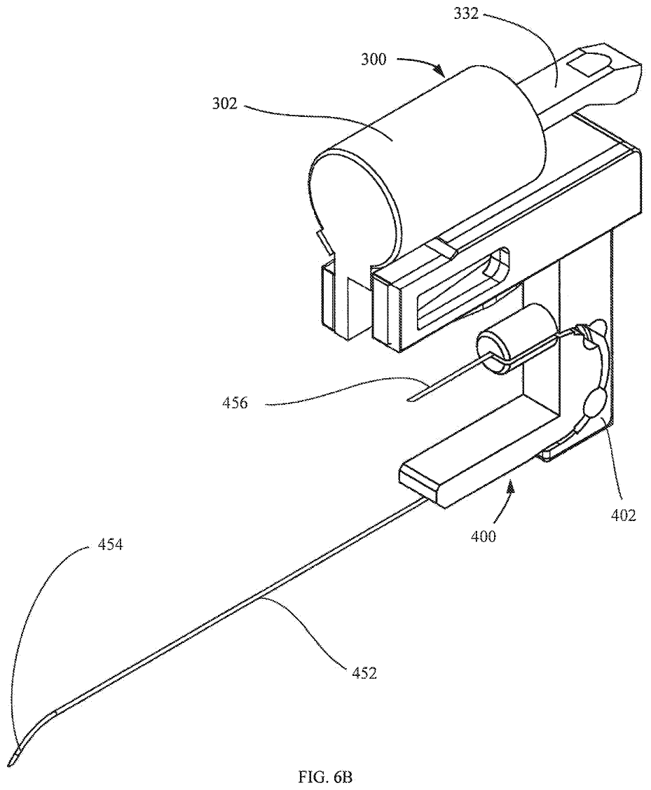

Referring now to FIG. 6A, the needle subassembly 400 is made up of a structure 402, which may be made of injected polymer, or any other suitable material. The needle subassembly structure 402 holds the needle 452, which has an injection end 454 through which fluid is injected into a living organism, and an extraction end 456 into which the fluid is extracted from the reservoir. The structure 402 has the appropriate shape to be guided and oriented in the mechanism 100 and to provide the latch and locking features for operation of the mechanism 100. The needle 452 is made of stainless steel, or any other suitable material.

Referring now to FIG. 6B to 6D, the insertion subassembly 300 is attached to the needle subassembly 400 prior to beginning the operational cycle of the mechanism 100. The insertion subassembly 300 is made up of a structure 302, which may be made of injected polymer, or any other suitable material. The structure 302 contains the insertion spring 352, which is compressed prior to device operation (e.g., at assembly). The spring may be made of stainless steel or any other suitable material. The structure 302 has the appropriate shape to be guided in the mechanism 100 and to provide the latch and locking features 332, 334 for operation of the device. The structure also includes a release feature 336 for releasing the needle subassembly 400 from the insertion subassembly 300 at step 10 of the operation of the mechanism 100.

Referring now to FIGS. 7A and 7B, the dispensing assembly 500 contains the reservoir subassembly 600. The dispensing assembly 500 is made up of a structure 502 and an intermediate part 512, which may be made of injected polymer, or any other suitable material. The structure 502 and the intermediate part 512 have the appropriate shape for guiding the mechanism 100 relative to adjacent components, and to provide the latch, locking and triggering features 504, 506, 514 for operation of the mechanism 100. The reservoir subassembly 600 is made up of a container 602, a septum 604, and contains the fluid 606 intended to be injected in the living organism by the operation of the device 1. The reservoir container 602 may be made of glass, or any other suitable material. The septum 604 is made of rubber-like, elastic, self-sealing material, or any other suitable material, and includes the necessary features so as to be able to be pierced by the needle extraction end 456 and to sealingly slide into the container 602 to act as a piston as required at the step 7 of the operation of the mechanism 100. Features integrated in the septum 604 to ensure the intended behavior may include a rigid or semi-rigid structure or a composite of such structures.

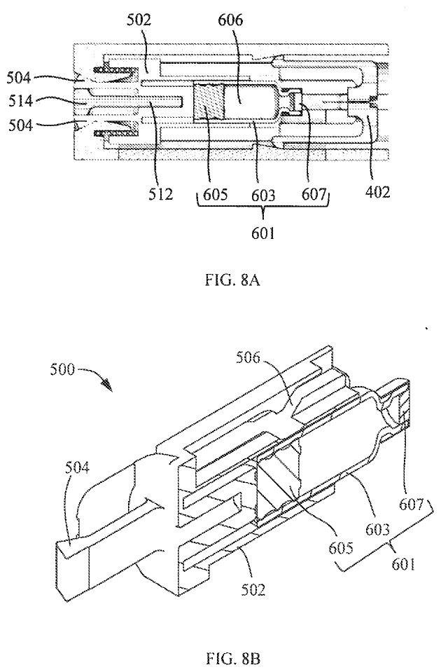

Referring now to FIGS. 8A to 8B, in an alternative embodiment, instead of the custom reservoir 600, the device contains a standard cartridge 601 as are readily available on the market. The cartridge 601 is made up of a container 603, a stopper 605 which act as a piston to fill the cartridge on the fill & finish lines (not represented), and a septum 607. The cartridge 601 contains the fluid 606 to be injected into the living organism. At step 7, the stopper 605 is to be used as a piston to expel the fluid 606 to be injected, while the septum 607 is pierced by the needle extraction end 456.

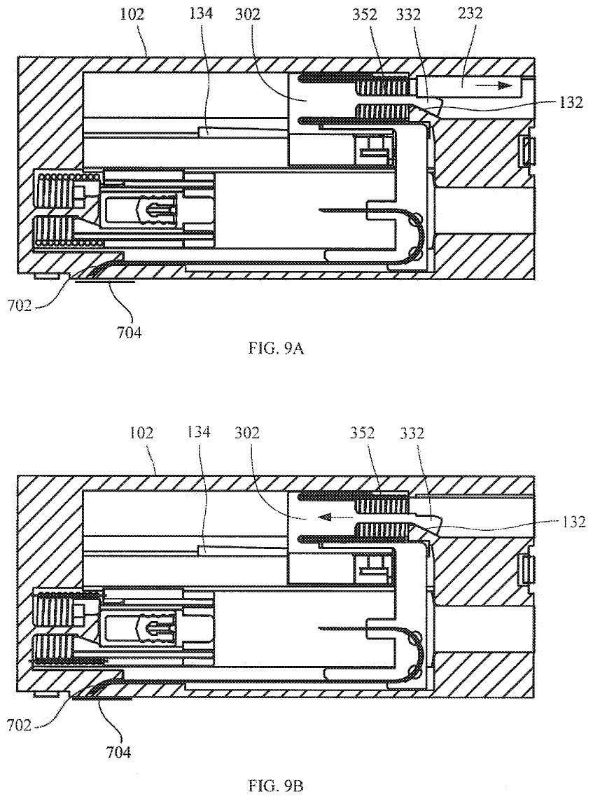

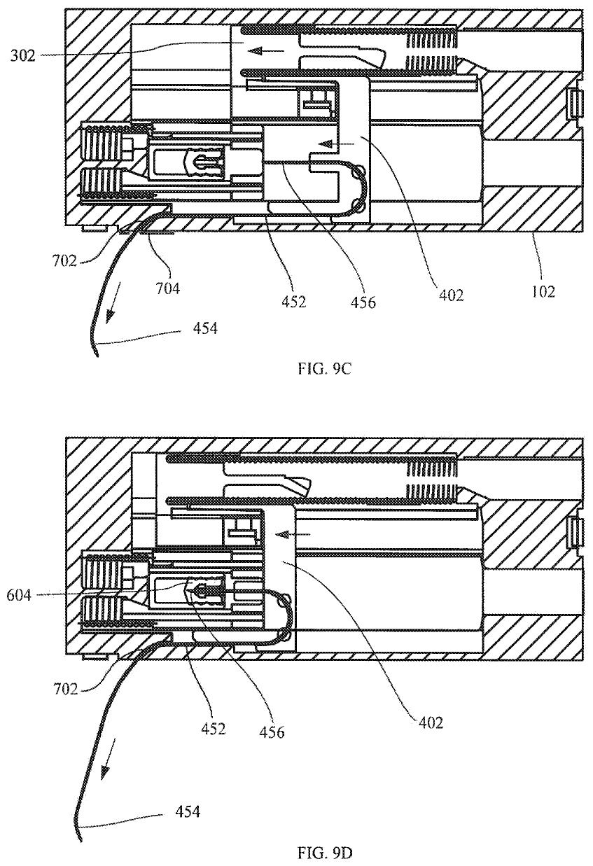

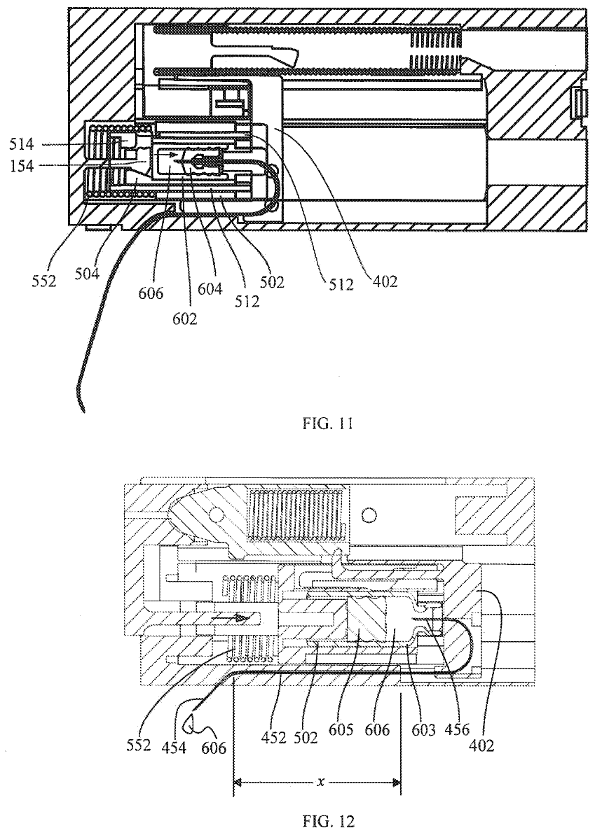

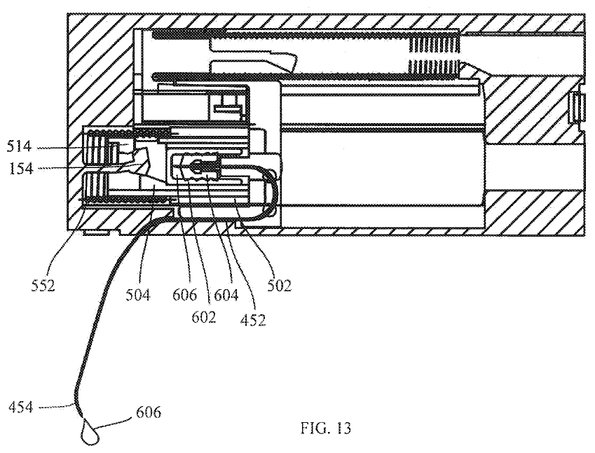

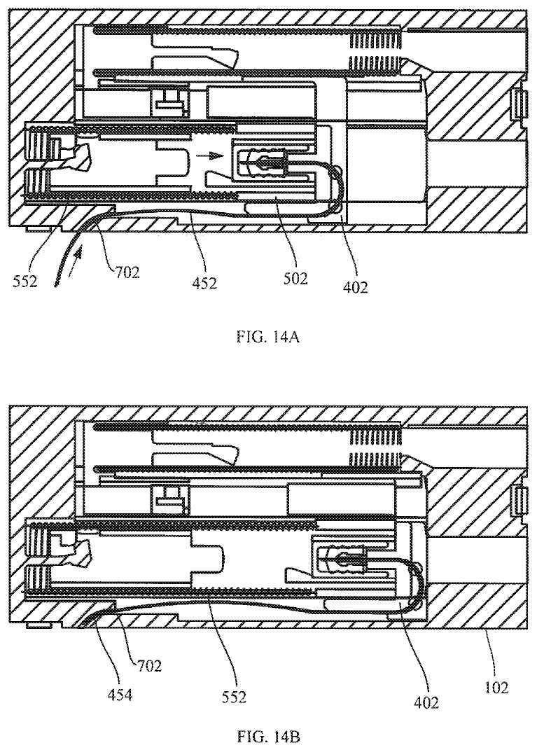

The sequence of operation of the device is as follows:
1. In case the device 1 is attached to a second device 1 as a pair (see FIG. 2), the user detaches the devices 1 in order to be able to use one device at a time.
2. The user removes the cover 4 (FIGS. 3 and 4).
3. The user applies the device body 2 against the skin of the living organism.
4. The pressure applied against the skin of the living organism is increased until a stable juxtaposition against the skin is attained for injection or until sufficient so that an optional detector 12 releases the push button 14 (FIGS. 3 and 4—at some predefined level of pressure). Alternatively the optional detector 12 actuates the trigger mechanism 200, in which case, we jump to step 6.
5. The user pushes the push button 14, actuating the trigger mechanism 200.
6. The trigger mechanism 200 releases the insertion subassembly 300 (FIG. 9A).
7. The insertion subassembly 300 pushes the needle assembly 400, until the needle injecting end 454 is able to insert into the living organism to an injection depth, and the needle extraction end 456 has pierced the reservoir septum 604 (FIGS. 9B to 9D).
8. Upon reaching this position, the insertion subassembly 300 is locked to the main structure 102 by a latch system 334, 134. As a consequence, the needle subassembly 400, which is still attached to the insertion subassembly 300, is also locked in place. At the same time, the dispensing assembly 500 is released, pushing the fluid reservoir subassembly 600 against the needle subassembly 400 (FIGS. 11 and 12).
9. The septum 604 is displaced towards the bottom of the fluid reservoir 602, acting as a piston and dispensing the fluid 606 through the needle extraction end 456, through the needle 452, through the needle injection end 454 into the living organism (FIG. 13).
10. Optionally, after the dispensing subassembly 500 has emptied the reservoir, the needle subassembly 400 is released from the insertion subassembly 300, and is pushed back to its start position, retracting the needle 452 until the needle injection end 454 is fully retracted within the main structure 102 (FIGS. 14A and 14B).

The combination of the herein described device and operating sequence brings the key advantage of optimizing the use of 2 sources of energy contained in the device (2 springs) using their maximum energy (when the springs are compressed at their maximum) to provide 2 bursts of energy: the first burst that is necessary to pierce the skin of the living organism and insert the needle to the injection site, the second burst to pump the fluid out of the reservoir and into the living organism. Retraction of the needle is provided by the remainder of energy of the second energy source.

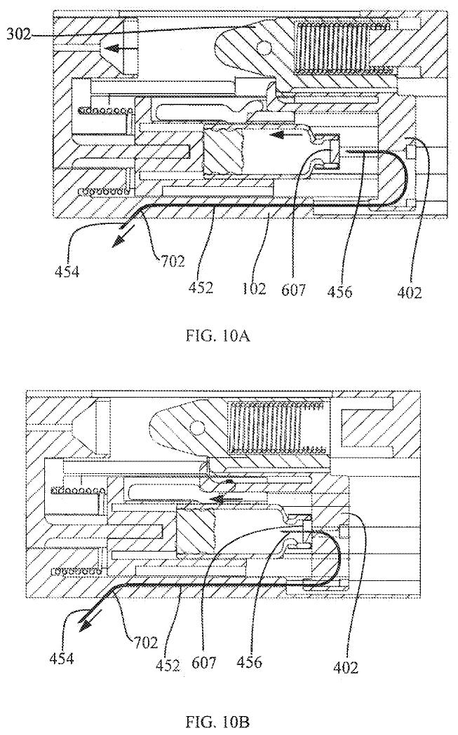

The following FIGS. 9A to 9D and 11 to 14B show more details about the first embodiment of the mechanism of the device 1 according to the invention FIGS. 10A to 10B and 12 show details of the second embodiment of the invention.

Referring now to FIG. 9A, referring to step 6 of operation of the mechanism 100, the release of the insertion subassembly 300 by the trigger mechanism 200 is represented as the removal of a locking element 232 from a latch feature made up of an appropriate shape (latch 132 in the main structure 102) and another corresponding shape in the insertion subassembly structure 302 (latch 332 in the insertion subassembly).

Referring now to FIGS. 9B to 9D, referring to step 7 of operation of the mechanism 100, the insertion subassembly 300 starts moving as pushed by the insertion spring 352. Since the needle subassembly 400 is attached to the insertion subassembly 300, the needle subassembly 400 executes the same movement, bringing the extraction end 456 of the needle 452 to pierce the septum 604 (see in particular FIG. 9D) and the injection end 454 of the needle 452 to pierce the skin of the living organism penetrating to the injection depth under the skin of the living organism.

Referring now to FIGS. 10A and 10B, in the second embodiment, the needle extraction end 456 pierces the cartridge's septum 607 (as opposed to the reservoir's septum 604 in the above embodiment). In the embodiment as described in the instant paragraph, the injection depth is not “under the skin” (as in the embodiment of the precedent paragraph), but “in or under the skin” of the living organism, such as in subcutaneous depth which may be at 1-2 mm under the surface of the skin.

Referring now to FIG. 11, in step 8 of operation of the mechanism 100, upon arrival, the needle subassembly structure 402 slides the intermediate part 512 of the dispensing assembly 500, opening the latch which is made up of feature 514 of the intermediate part 512, a latch feature 154 of the main structure 102 and a feature 504 of dispensing assembly structure 502. At this stage, the fluidic connection from the fluid container 602 through the needle extraction end 456 through the needle 452 through the needle injection end 454 is open, and the spring 552 of the dispensing subassembly is released. At the same time, the locking feature 334 (shown in FIG. 6C) snaps in the corresponding feature 134 of the main structure 102, immobilizing the insertion subassembly 300 and the attached needle assembly 400 so that they can't move backwards.

Referring now to FIG. 12, in the second embodiment, the dispensing assembly structure 502 moves relative to the cartridge's container 603, pushing the stopper 605 inside the container 603, expelling the fluid 606 outside of the container 603 through the needle extraction end 456, through the needle 452, through the needle injection end 454 into the living organism.

Note that dimension x (shown in FIG. 12) or x′ (shown in FIG. 16) can be selected in the design phase of all embodiments shown herein, from 1 mm or less to 40 mm or more, depending on the application needs.

Referring now to FIG. 13, in step 9 of operation of the mechanism 100, when released, the dispensing spring 552 pushes the reservoir container 602 against the needle subassembly structure 402, which generates a relative movement of the septum 604 towards the bottom of the container 602, pushing the fluid 606 out of the container 602 through the needle extraction end 456, through the needle 452, through the needle injection end 454 into the living organism.

Referring now to FIGS. 14A and 14B, in optional step 10 of operation of the mechanism 100, when the dispensing spring 552 which pushes the reservoir container 602 against the needle subassembly structure 402 reaches the point where the triggering feature 506 of the dispensing subassembly structure 502 pushes the release feature 336, the needle subassembly 400 is released from the insertion subassembly 300 and starts moving back to the position it had prior to the start of the operation. While moving backward, the needle subassembly 400, pushed by the dispensing subassembly 500, retracts the needle 452 until the needle injection end 454 is fully retracted within the main structure 102.

Note that the needle 452, optionally passes through a mandrel 702 that deforms the needle so as to prevent it from being reused. In one embodiment, the needle is deformed so as to be bowed, thus ensuring that buckling of the needle because it is no longer straight prevents it from passing through the mandrel once again.

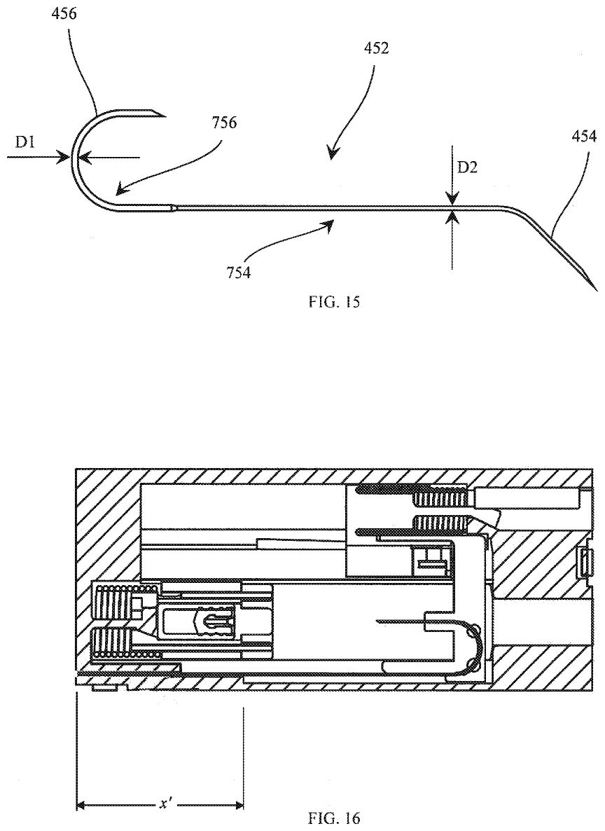

Referring now to FIG. 15, showing some further details of the needle 452. The first end of the needle, also referred to as needle extraction end 456 is shown in more detail. Further, the second end of the needle, also referred to as needle injection end 454 is shown in more detail. The needle 452 comprises an approx. 180° turn in the area of the first end of the needle. The diameter D1 of the needle in that area may be of a certain size, while the diameter D2 of the other portion of the needle may be larger, smaller or the same as D1. The portion with diameter D1 remains during operation inside the housing 2, while the portion of the needle with diameter D2 is configured to bend and at least partly inserted into the patient during operation. The portion of the needle with diameter D2 is on the side of needle's injection end 454. The needle 452 comprises an interior channel (not shown) going from one end to the other end of the needle. The diameter of the channel may be adapted to the fluid's viscosity. However, the energy of one or both energy sources may be adapted to the fluid's viscosity as well. In an even more preferred way, both the size of the diameter of the channel and the energy of one or both energy sources are adapted to the fluid's viscosity. In order to ensure deformation and buckling of the needle though the mandrel during operation, the diameter of the channel and the diameter D2 are selected so as to ensure proper deformation (bending and/or buckling). In a further embodiment, the needle may have varying diameter D2 to ensure proper bending and buckling. In another further embodiment, the channel may comprise a restrictor, such as a flow restrictor.

By placing and affixing or integrating into one housing one or more devices shown in FIGS. 5A-5B, 6A-6D, 7A-7B, 8A-8B, 9A-9D, 10A-10B, 11-13, 14A-14B, and 16 (such as adjacent or parallel to one another), the embodiments as described in the present description may be configured to accommodate more than one needle 454 in parallel (simultaneously, optionally side-by-side), such that the first ends 456 of these needles pierce the same septum or different septi for the same or different reservoirs. In a further alternative, the embodiments as described in the present description may be configured to handle more than one needle 454 in parallel, such that the first ends 456 of these needles pierce at least two septi, each septum being allocated to an individual reservoir. Using more than one needle simultaneously provides several advantages:
it allows for using smaller needles which generate less pain when being inserted into the body of the living organism;
it allows for simultaneously injecting several smaller doses of fluid into a living organism instead of one larger dose thereby generating less local tissue damage and therefore reducing the pain of the injection for the patient; and
it allows for injecting the fluid at multiple locations thereby providing more efficient treatment of the patient, as the injected fluid will diffuse easier and faster within the body of the living organism.

Referring now to FIG. 16, an alternative embodiment is shown, wherein the needle's injection end 454 is straight and remains straight during operation. For operation, the user applies the device body against the skin of the living organism such that the injection end of the needle 454 enters substantially orthogonal into the living organism. In this embodiment, the needle is not bent during operation. In addition, retraction of the needle may be accomplished by the user pulling the device away from the skin, as in a standard syringe.

Note that all mobile elements of the mechanism 100 subject to relative sliding or frictional forces may have a coating applied to their surfaces where appropriate to ensure low friction.

The fluid for injection is typically a fluid selected from a group of fluids consisting of peptides, proteins, hormones including insulin, calcitonin, calcitonin gene regulating protein, atrial natriuretic protein, colony stimulating factor, betaseron, erythropoietin (EPO), interferons such as .alpha., .beta. or .gamma. interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-Ira; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, verapamil, isosorbide-5-monotritate, organic nitrates, agents used in treatment of heart disorders, and analogues thereof; sedatives such as benzodiazepines, phenothiazines, and analogues thereof; chelating agents such as defroxanune, and analogues thereof; anti-diuretic agents such as desmopressin, vasopressin, and analogues thereof; anti-anginal agents such as fluorouracil, bleomycin, and analogues thereof; anti-neoplastics such as fluorouracil, bleomycin, and analogues thereof; prostaglandins and analogues thereof, and chemotherapy agents such as vincristine, and analogues thereof, treatments for attention deficit disorder, methylphenidate, fluvoxamine, bisoprolol, tacrolimus, sacrolimus and cyclosporin, vitamins suspended in a liquid carrier, antivenoms, syrums, medications, antibodies, Actemra (tocilizumab), Adcretris (brentuximab vedotin), Arzerra (ofatumumab), Avastin (bevacizumab), Benlysta (belimumab), Cimzia (certolizumab pegol), Erbitux (cetuximab), Herceptin (trastuzumab), Humira (adalimumab), Ilaris (canakinumab), Lucentis (ranibizumab), Mylotarg (gemtuzumab ozogamicin), Perjeta (pertuzumab), Prolia (denosumab), Remicade (infliximab), Simponi (golimumab), Soliris (eculizumab), Stelara (ustekinumab), Tysabri (natalizumab), Vectibix (panitumumab). Xgeva (denosumab), Xolair (omalizumab), Yervoy (ipilimumab), and Zevalin (ibritumomab tiuxetan).

In an advantage, the invention provides an easy to use, user friendly device that allows any person, even the patients themselves, to perform an injection of a fluid, of particular interest, a COVID-19 vaccine, without the intervention of medical personnel, thereby reducing the burden on world healthcare systems.

It is another object of the invention to provide a user friendly device that allows a patient to inject themselves without requiring him to make an aggressive gesture like stabbing himself.

In an advantage, the invention provides a device that can step up to the challenge of new molecular therapeutic innovations such as high viscosity fluids whose characteristics otherwise limit administration options.

In an advantage, the invention provides a device that can simultaneously inject several smaller doses of fluid into a living organism instead of one larger dose, generating less local tissue damage and therefore reducing the pain of the injection for the patient.

In an advantage, the invention provides a device with and extended product life cycle.

In an advantage, the invention provides a device that retracts the needle to prevent inadvertent harm that might be caused by an exposed needle.

In an advantage, the invention provides the option of injecting at an angle as the needle passes through a mandrel.

In an advantage, the invention helps prevent unsanitary or perhaps inadvertent reuse of the device, thus providing a safer means of injecting a fluid.

The invention may be summarized as follows:

1. A fluid dispensing device 1 adapted for intramuscular and/or subcutaneous injection into a living organism, the device having an energy storage arrangement 352, 552 enclosed in a housing 2, the energy storage arrangement providing energy in at least two bursts, the first burst being that which is necessary to pierce the skin of the living organism and insert at least one needle 454 to the injection site, the second burst being that which is necessary to press the fluid out of the at least one reservoir 602 and into the living organism, and, optionally, to retract the at least one needle out of the living organism.
2. The device of feature set 1, wherein the first burst is provided by a first energy source 352 and the second burst is provided by a second energy source 552.
3. The device of any of the above feature sets, wherein the energy storage devices 352, 552 are springs.
4. The device of any of the above feature sets, wherein the at least one needle 452 has first and second ends which are adapted to pierce respective surfaces while translating together in a direction such that when used, the direction of movement of the first end of the at least one needle is parallel to a patient's skin and the direction of movement of the second end of the at least one needle is substantially non orthogonal to the patient's skin.
5. The device of any of the above feature sets, wherein the at least one needle passes through a mandrel which diverts the direction of translation of the at least one needle from one orientation, typically parallel, with respect to the patient's skin to a non-orthogonal angle with respect to the patient's skin.
6. The device of the above feature set wherein the mandrel 702 is adapted to deform the needle 452 as it passes therethrough.
7. The device of the above feature set, wherein the mandel 702 is adapted to deform the needle beyond the needle's elastic limit thereby preventing re-use of the device.
8. The device of any of the above feature sets, wherein at least one thin, impermeable membrane 704 is adhesively attached to the housing 2 so as to cover and seal an at least one opening 706 in the device through which the at least one needle pierces and extends upon actuation for injection.
9. A method of operation of the device 1 includes the following steps:
a. in case the device 1 is attached to a second device 1 as a pair, detaching the devices 1 to enable use one device at a time;
b. removing a cover 4 unless a thin membrane 704, which the at least one needle 454 can pierce, is used to seal the device, in which case no cover is required;
c. applying the device body 2 with pressure against the skin of the living organism;
d. optionally increasing the pressure applied against the skin of the living organism until it is sufficient so that the optional detector 12 releases the push button 14 allowing manual triggering in the next step or alternatively, the detector 12 automatically actuating the trigger mechanism 200, in which latter case, skip to step f;
e. pushing the push button 14, thereby actuating the trigger mechanism 200 which in turn releases the at least one insertion subassembly 300;
f. pushing an at least one needle subassembly 400 using the insertion subassembly 300 until the needle injecting end 454 is inserted into the living organism at the injection depth, and the needle extraction end 456 has pierced an at least one septum 604 of the at least one reservoir 602;
h. upon reaching this position, locking the insertion subassembly 300 to the main structure 102;
i. locking the at least one needle subassembly 400, which is still attached to the at least one insertion subassembly 300, in place, while, at the same time, releasing the dispensing assembly 500, pushing the fluid reservoir subassembly 600 against the needle subassembly 400
j. displacing the at least one septum 604 towards the bottom of the at least one fluid reservoir 602, acting as a piston and dispensing the fluid 606 through the needle extraction end 456, through the at least one needle 452, through the at least one needle injection end 454 into the living organism; and
k. removing the device from the living organism.
   10. The method of feature set 9, wherein just prior to removal in step k, when the dispensing subassembly 500 has emptied the at least one reservoir, the method includes the additional step of releasing the at least one needle subassembly 400 from the at least one insertion subassembly 300, which is then pushed back to its start position, thereby retracting the at least one needle 452 until the needle injection end 454 is fully retracted within the main structure 102 thereby preventing harm that might be caused by an exposed needle.
   11. The method of operation of feature set 1, wherein the locking in step h is accomplished using a latch system (334).
   12. The device of any of the above feature sets, wherein at least a portion (754) of the interior channel of the needle (452) conducting the fluid has a diameter different from that of another portion (756) of the interior channel of the needle.
   13. The device of any of the above feature sets, wherein at least two needles within one housing are used simultaneously to conduct the fluid into the living organism.

Further, the invention should be considered as comprising all possible combinations of every feature described in the instant specification, appended feature sets, and/or drawing figures which may be considered new, inventive and industrially applicable.

It should be appreciated that the particular implementations shown and herein described are representative of the invention and its best mode and are not intended to limit the scope of the present invention in any way.

As will be appreciated by skilled artisans, the present invention may be embodied as a system, a device, or a method.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto. Such claims are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

Addendum

Following elements are disclosed in FIGS. 1 to 3:
1 Dispensing device
2 Body of the dispensing device/housing
4 Cover
12 Detector of patient's skin presence (optional)/pressure-sensitive safety release mechanism
14 Trigger button
16 Indicator device ready to use/used

Following elements are disclosed in FIGS. 4 to 14B:
100 main mechanism
102 main structure
132 latch element
134 protrusion
154 latch element
200 trigger mechanism
232 latch element
300 insertion subassembly
302 insertion subassembly structure
332 latch element
334 latch element
336 latch release lever
352 insertion spring/energy source
400 needle subassembly
402 needle subassembly structure
452 needle
454 needle injection end
456 needle extraction end
500 dispensing subassembly
502 dispensing subassembly structure
504 latch element
506 protrusion
512 intermediate part
514 latch element
552 dispensing and retraction spring/energy source
600 reservoir subassembly
601 cartridge (standard cartridge or off the shelf cartridge)
602 reservoir body
604 septum
606 fluid
702 mandrel
704 impermeable membrane
706 opening
754 portion of the interior channel
756 portion of the interior channel

1. A fluid dispensing device adapted for intramuscular and/or subcutaneous injection into a living organism, the device having an energy storage arrangement enclosed in a housing, the energy storage arrangement providing energy in at least two bursts, the first burst being that which is necessary to pierce the skin of the living organism and insert at least one needle to the injection site, the second burst being that which is necessary to press the fluid out of the at least one reservoir and into the living organism, and, optionally, to retract the at least one needle out of the living organism.

2. The device of claim 1, wherein the first burst is provided by a first energy source and the second burst is provided by a second energy source.

3. The device of any of the above claims, wherein the energy storage devices are springs.

4. The device of any of the above claims, wherein the at least one needle has first and second ends which are adapted to, pierce respective surfaces while translating together in a direction such that when used, the direction of movement of the first end of the at least one needle is parallel to a patient's skin and the direction of movement of the second end of the at least one needle is substantially non orthogonal to the patient's skin.

5. The device of any of the above claims, wherein the at least one needle passes through a mandrel which diverts the direction of translation of the at least one needle from one orientation, typically parallel, with respect to the patient's skin to a non-orthogonal angle with respect to the patient's skin.

6. The device of the above claim wherein the mandrel is adapted to deform the needle as it passes therethrough.

7. The device of claim 6, wherein the mandel is adapted to deform the needle beyond the needle's elastic limit thereby preventing re-use of the device.

8. The device of claim 1, wherein at least one thin, impermeable membrane is adhesively attached to the housing so as to cover and seal an at least one opening in the device through which the at least one needle pierces and extends upon actuation for injection.

9. A method of operation of the device includes the following steps:
a) in case the device is attached to a second device as a pair, detaching the devices (1) to enable use one device at a time;
b) removing a cover unless a thin membrane, which the at least one needle can pierce, is used to seal the device, in which case no cover is required;
c) applying the device body with pressure against the skin of the living organism;
d) optionally increasing the pressure applied against the skin of the living organism until it is sufficient so that the optional detector releases the push button (14) allowing manual triggering in the next step or alternatively, the detector automatically actuating the trigger mechanism, in which latter case, skip to step f;
e) pushing the push button, thereby actuating the trigger mechanism (200) which in turn releases the at least one insertion subassembly;
f) pushing an at least one needle subassembly using the insertion subassembly until the needle injecting end is inserted into the living organism at the injection depth, and the needle extraction end has pierced an at least one septum of the at least one reservoir;
h) upon reaching this position, locking the insertion subassembly to the main structure (602);
i) locking the at least one needle subassembly, which is still attached to the at least one insertion subassembly, in place, while, at the same time, releasing the dispensing assembly, pushing the fluid reservoir subassembly against the needle subassembly;
j) displacing the at least one septum towards the bottom of the at least one fluid reservoir, acting as a piston and dispensing the fluid through the needle extraction end through the at least one needle, through the at least one needle injection end into the living organism; and
k) removing the device from the living organism.

10. The method of claim 9, wherein just prior to removal in step k, when the dispensing subassembly has emptied the at least one reservoir, the method includes the additional step of releasing the at least one needle subassembly from the at least one insertion subassembly, which is then pushed back to its start position, thereby retracting the at least one needle until the needle injection end is fully retracted within the main structure thereby preventing harm that might be caused by an exposed needle.

11. The method of operation of claim 1, wherein the locking in step h is accomplished using a latch system.

12. The device of any of the above claims, wherein at least a portion of the interior channel of the needle conducting the fluid has a diameter different from that of another portion of the interior channel of the needle.

13. The device of any of the above claims, wherein at least two needles within one housing are used simultaneously to conduct the fluid into the living organism.
